(19) United States
(12) Patent Application Publication
(10) Pub. No.: US 2018/0147416 A1
(43) Pub. Date: May 31, 2018

(54) Method and System for Appetite Suppression by Laser Stimulation of Acupuncture Points

(71) Applicant: LaserStim, Inc., Plano, TX (US)

(72) Inventors: Kim Robin Segel, Plano, TX (US); Jeff Adelglass, Plano, TX (US)

(73) Assignee: LaserStim, Inc., Plano, TX (US)

(21) Appl. No.: 15/816,878

(22) Filed: Nov. 17, 2017

Related U.S. Application Data
(60) Provisional application No. 62/423,691, filed on Nov. 17, 2016.

(51) Int. Cl. A61N 5/06; A61H 39/00

(52) U.S. Cl. CPC A61N 5/0619; A61H 39/00; A61N 2005/067; A61N 2005/0663; A61N 2005/0647

(57) ABSTRACT

The invention provides a method and system for suppressing appetite. An illumination device such as a lamp or helmet is placed over the patient's head, the illumination device having a plurality of light sources in a fixed configuration selected to illuminate the patient's head with blue light having a wavelength in the range of 400 nm-495 nm. Concurrently with illuminating the patient's head with blue light, at least one acupuncture point on the patient is irradiated with coherent laser light having a wavelength ranging from 400 nm-2500 nm at a power level of 500 mw or less. The laser light may be supplied by a hand held wand, a face cover, and/or ear covers each having laser diodes held in contact with the respective acupuncture points.

### CROSS-REFERENCE TO RELATED APPLICATIONS

This application claims the benefit of and priority to U.S. Provisional Patent Application No. 62/423,691, filed Nov. 17, 2016, the technical disclosures of which are hereby incorporated herein by reference.

### TECHNICAL FIELD

The present invention relates generally to the treatment of living biological tissue by optical irradiation, and in particular to a system for suppressing appetite by stimulating acupuncture points with laser irradiation.

### BACKGROUND OF THE INVENTION

Various methods have been employed to suppress appetite for the purposes of weight loss. The vast majority of approaches are pharmacological in nature, employing chemical compounds that work on the appetite control centers of the brain. In more recent years surgical methods have also been introduced such as gastric bypass, which seek to reduce appetite by physically reducing the amount of food a person can eat before feeling full. However, both pharmacological and surgical methods for reducing appetite also carry with them significant side effects and complications, some of which are potentially life threatening. They also entail considerable financial costs.

Another recent approach (albeit with ancient roots) is the use of acupuncture for controlling appetite. Acupuncture is an ancient method of treating physical disorders by inserting tiny needles into key points of the body to stimulate a desired physiological response, either local or systemic, e.g., relaxation, pain reduction, improved respiration, etc. Although the exact physiological mechanism by which acupuncture works is still the subject of much debate, its effectiveness in many applications has been demonstrating in many studies over the years.

While generally free of known side effects, many people are hesitant to use acupuncture due to fear of needles. However, recent research has indicated that lasers may be used to stimulate acupuncture points along the body with results comparable to the use of needles.

Optical energy generated by lasers has been used for various medical and surgical purposes because laser light, as a result of its monochromatic and coherent nature, can be selectively absorbed by living tissue. The absorption of the optical energy from laser light depends upon certain characteristics of the wavelength of the light and properties of the irradiated tissue, including reflectivity, absorption coefficient, scattering coefficient, thermal conductivity, and thermal diffusion constant. The reflectivity, absorption coefficient, and scattering coefficient are dependent upon the wavelength of the optical radiation. The absorption coefficient is known to depend upon such factors as interband transition, free electron absorption, grid absorption (photon absorption), and impurity absorption, which are also dependent upon the wavelength of the optical radiation.

In living tissue, water is a predominant component and has, in the infrared portion of the electromagnetic spectrum, an absorption band determined by the vibration of water molecules. In the visible portion of the spectrum, there exists absorption due to the presence of hemoglobin. Further, the scattering coefficient in living tissue is a dominant factor.

Thus, for a given tissue type, the laser light may propagate through the tissue substantially unattenuated, or may be almost entirely absorbed. It is generally preferred that the laser light be essentially transmissive through tissues which are not to be affected, and absorbed by tissues which are to be affected. For example, when applying laser radiation to a region of tissue permeated with water or blood, it is desired that the optical energy not be absorbed by the water or blood, thereby permitting the laser energy to be directed specifically to the tissue to be treated. Another advantage of laser treatment is that the optical energy can be delivered to the treatment tissues in a precise, well-defined location such as an acupuncture point and at predetermined, limited energy levels.

### SUMMARY OF THE INVENTION

The invention provides a method and system for suppressing appetite. An illumination device such as a lamp or helmet is placed over the patient's head, the illumination device having a plurality of light sources in a fixed configuration selected to illuminate the patient's head with blue light having a wavelength in the range of 400 nm-495 nm. Concurrently with illuminating the patient's head with blue light, at least one acupuncture point on the patient is irradiated with coherent laser light having a wavelength ranging from 400 nm-2500 nm at a power level of 500 mw or less. The laser light may be supplied by a hand held wand, a face cover, and/or ear covers each having laser diodes held in contact with the respective acupuncture points.

### BRIEF DESCRIPTION OF THE DRAWINGS

The novel features believed characteristic of the invention are set forth in the appended claims. The invention itself, however, as well as a preferred mode of use, further objects and advantages thereof, will best be understood by reference to the following detailed description of an illustrative embodiment when read in conjunction with the accompanying drawings, wherein:

FIG. 1 shows a schematic diagram of a diode laser irradiation system of the present invention;

FIG. 2 shows an elevational view of a wand used in the system of FIG. 1;

FIG. 3A shows an enlarged, elevational view of a laser resonator used in the wand of FIG. 2;

FIG. 3B shows an enlarged, end view of the laser resonator used in the wand of FIG. 3A;

FIG. 4 illustrates an illumination helmet that is used in conjunction with laser stimulation of acupuncture/acupressure points;

FIG. 5A shows a development view of one form of cap showing the placement of the lasers for one representative embodiment, according to embodiments of the invention;

FIG. 5B shows a development view of another form of cap illustrating the placement of the lasers for another representative embodiment, according to embodiments of the invention; and

FIG. 6 shows a side view of the cap given in FIG. 5, according to an embodiment of the invention;

FIG. 7 shows an alternate embodiment of the illumination helmet;

FIG. 8 shows a blue light lamp in accordance with an embodiment of the invention;

FIG. 9 shows ear covers containing laser diodes for stimulating acupuncture points on the ears in accordance with an embodiment of the invention;

FIG. 10 illustrates the acupuncture/acupressure points on the face and head that are stimulated to suppress appetite in accordance with the invention;

FIG. 11A is a top view of a face cover that stimulates acupuncture points on the face in accordance with the invention; and

FIG. 11B shows in the inside facing surface of the face cover.

### DETAILED DESCRIPTION OF THE DRAWINGS

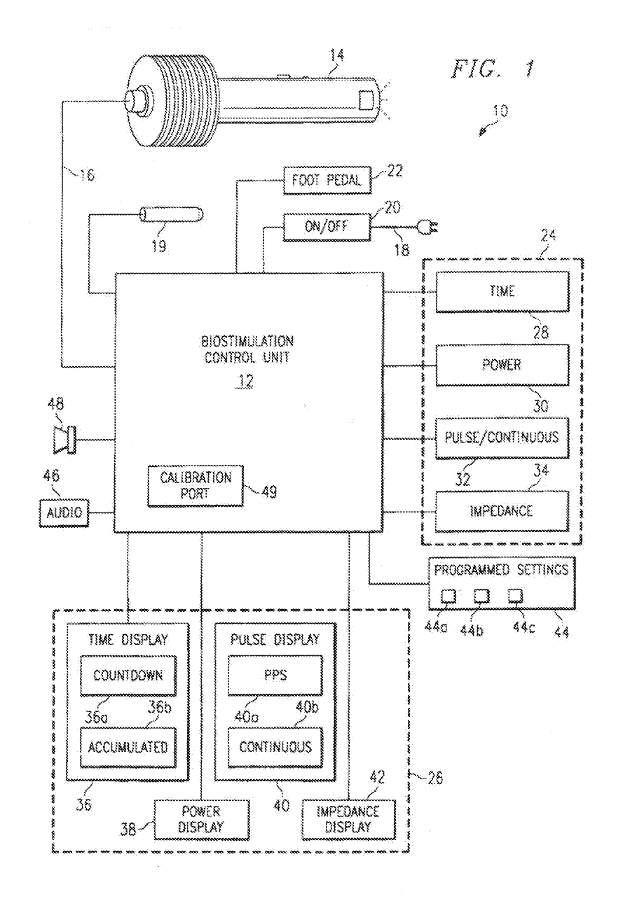

Referring to FIG. 1, the reference numeral 10 refers generally to the diode laser irradiation system of the present invention which includes a biostimulation control unit 12 for controlling the operation of a hand-operated probe, i.e., a laser treatment wand 14, electrically connected to the control unit via a coaxial cable 16. As will be described in detail below, the wand 14 houses a diode laser capable of emitting low level reactive laser light for use in tissue irradiation therapy.

The control unit 12 receives power through a power supply line 18 adapted for connection to a conventional 120-volt power outlet. A ground piece 19 is connected to the control unit 12 and is held by a patient receiving the tissue irradiation therapy to provide an electrical ground for safety purposes. An on/off switch 20 is connected in series with the line 18 for controlling the flow of power through the line. A foot pedal 22 is connected to the control unit 12 and is depressible for activating the generation and emission of laser light from the wand 14. Activation may alternatively, or additionally, be provided using a switch on the wand 14.

The control unit 12 includes laser setting controls 24 and corresponding setting displays 26. The setting controls 24 are utilized to select operational parameters of the control unit 12 to effect the rate of absorption of tissue irradiated by the wand 14, according to desired treatment protocols. Generally, the treatment protocols provide for a rate of absorption and conversion to heat in the irradiated tissue in a range between a minimum rate sufficient to elevate the average temperature of the irradiated tissue to a level above the basal body temperature of the subject and a maximum rate which is less than the rate at which the irradiated tissue is converted to a collagenous substance. The treatment protocols vary time, power, and pulse/continuous mode parameters in order to achieve the desired therapeutic effects.

The setting controls 24 include a treatment time control 28, a power control 30, and a pulse/continuous mode control 32. Adjustments in treatment time, power and pulse/continuous mode operation of the wand 14 utilizing the controls 28-32 make possible improved therapeutic effects based upon the aforementioned treatment protocols involving one or more of these parameters. Also, an impedance control 34 is provided adjusting an impedance measurement of the tissue to a baseline value, according to skin resistance. It is understood that, according to the specific embodiment of the control unit 12, the setting controls 24 may include any combination of one or more of the controls 28-34.

The setting displays 26 include a time display 36, a power display 38, a pulse display 40 and an impedance display 42. In one embodiment, each of the displays 26 are light emitting diode (LED) displays such that the corresponding setting controls 24 can be operated to increment or decrement the settings, which are then indicated on the displays. A programmed settings control 44 is used to save setting selections and then automatically recall them for convenience, using one or more buttons 44a-44c, for example.

The time control 28 adjusts the time that laser light is emitted from the wand 14, as indicated on the time display 36. The time display 36 includes a countdown display 36a and an accumulated display 36b. Once the time control 28 is set, the countdown display 36a indicates the setting so that as the wand 14 is operated the time is decremented to zero. The accumulated time display 36b increments from zero (or any other reset value) as the wand 14 is operated so that the total treatment time is displayed. The time display 36 takes into account the pulsed or continuous mode operation of the system 10.

The power control 30 adjusts the power dissipation level of the laser light from the wand 14 in a range from zero to 1000 milliwatts (mw), with typical operation ranging up to about 500 mw. The pulse/continuous mode control 32 sets the system 10 to generate laser light energy from the wand 14 either continuously or as a series of pulses. The control 32 may include, for example, a pulse duration rheostat (not shown) for adjusting the pulse-on or pulse-off time of the wand 14. In one implementation, the pulses-per-second (PPS) is set in a range from zero to 9995, adjustable in 5 step increments. The PPS setting is displayed on a PPS display 40a. The pulse duration may alternatively, or additionally, be displayed indicating the duty cycle of pulses ranging from 5 to 99 (e.g., 5 meaning that the laser is “on” 5% of the time). A continuous mode display 40b is activated when the system 10 is being operated in the continuous wattage (CW) mode of operation.

An audio volume control 46 is provided for generating an audible warning tone from a speaker 48 when laser light is being generated. Thus, for example, the tone may be pulsed when the system is operating in the pulse mode of operation.

The impedance control 34 is a sensitivity setting that is calibrated and set, according to the tissue skin resistance, to a baseline value which is then indicated on the impedance display 42.

A calibration port 49 is utilized to verify laser performance by placing the wand 14 in front of the port and operating the system 10. The port 49 determines whether the system 10 is operating within calibration specifications and automatically adjusts the system parameters.

While not shown, the control unit 12 includes digital and analog electronic circuitry for implementing the foregoing features. The details of the electronic circuitry necessary to implement these features will be readily understood by one of ordinary skill in the art in conjunction with the present disclosure and therefore will not be described in further detail.

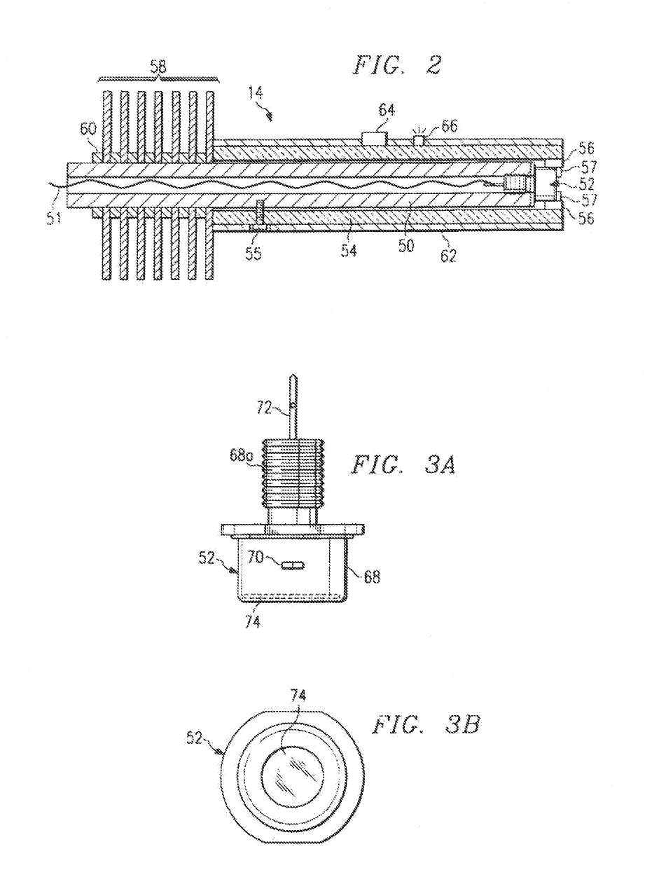

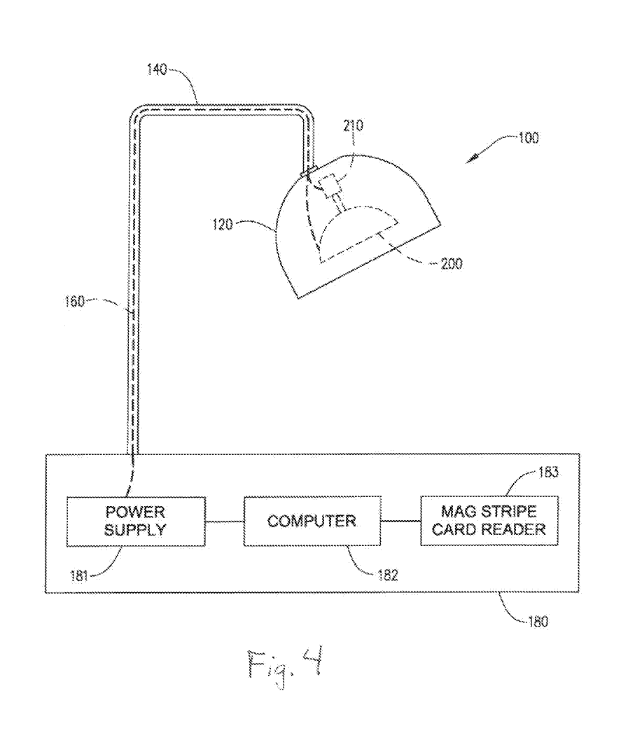

Referring to FIG. 2, the wand 14, sized to be easily manipulated by the user, includes a heat-conductive, metal bar 50. The bar 50 is hollow along its central axis and is threaded on its interior at a first end for receiving a laser resonator 52, described further below with reference to FIGS. 3A and 3B. Wiring 51 extends from the resonator 52 through the hollow axis of the bar 50 for connection to the coaxial cable 16 (FIG. 1). In the preferred embodiment the bar 50 is copper or steel and thus conducts electricity for providing a ground connection for the resonator 52 to the cable 16.

A glass noryl sleeve 54 is placed over the bar 50 for purposes of electrical and thermal insulation. A screw 55 extending through the sleeve 54 anchors the sleeve to the bar 50. As shown, the resonator 52 is recessed slightly within the sleeve 54. An impedance oring 56, formed of a conductive metal, is press-fitted into the end of the sleeve 54 so that when the wand 14 makes contact with tissue, the ring 56 touches the tissue. The ring 56 is electrically connected through the wand 14 to the unit 12. The ring 56 measures impedance by measuring angular DC resistance with an insulator ohmmeter, for example, of the tissue being irradiated by the wand 14 which is then displayed as impedance on the display 42. Any other suitable impedance measurement circuit may be utilized, as will be apparent to one skilled in the art.

A feedback sensor 57 is located in the end of the sleeve 54 for measuring the output of the resonator 52. While not shown, the sensor 57 is connected electronically to the control unit 12 and to a feedback circuit within the control unit. A small percentage of the diode laser light from the resonator 52 is thus detected by the sensor 57 and channeled into the feedback circuit of the control unit 12 to measure and control performance of the resonator. Out-of-specification temperature, power, pulse frequency or duration is thus corrected or the system 10 is automatically turned off.

Multiple metallic fins 58 are placed over the end of the bar 50 and are separated and held in place by spacers 60 press-fitted over the bar 50. The fins 58 act as a heat sink to absorb heat from the laser through the bar 50 and dissipate it into the surrounding air. The spacers 60 placed between each fin 58 enable air to flow between the fins, thereby providing for increased heat transfer from the wand 14.

A casing 62 fits over the sleeve 54 and serves as a hand grip and structure to support a switch 64 and light 66. The switch 64 is used to actuate the wand 14 by the operator wherein the switch must be depressed for the wand to operate. The switch 64 is wired in a suitable manner to the control unit 12 and is used either alone or in conjunction with the foot pedal 22. The light 66 is illuminated when the wand 14 is in operation.

As shown in FIG. 3A, the laser resonator 52 includes a housing 68 having threads 68a configured for matingly engaging the threaded portion of the bar 50 in its first end. A Gallium Arsenide (GaAs) or Indium-doped Gallium Arsenide (In:GaAs) semiconductor diode 70 is centrally positioned in the housing 68 facing in a direction outwardly from the housing 68, and is electrically connected for receiving electric current through the threads 68a and an electrode 72 connected to the wiring 51 that extends longitudinally through the hollow interior of the tube 50 (FIG. 2). The amount of Indium with which the Gallium Arsenide is doped in the diode 70 is an amount appropriate so that the diode 70, when electrically activated, generates, in the direction outwardly from the housing 68, low level reactive laser light having a power output level of 50-500 mw and a wavelength of approximately 1000-2500 nm in the near-infrared region of the electromagnetic spectrum. Other types of diode semiconductor lasers may also be used to produce the foregoing wavelengths, e.g., Helium Neon or the like.

Alternatively, diode 70 can be a Gallium Nitride (GaN) or Indium-doped Gallium Nitride (In:GaN) semiconductor diode having a power output level of 100-1000 mw, typically less than 500 mw and wavelength ranging from approximately 400-495 nm in the blue region of the electromagnetic spectrum. The blue-light laser diode can also be constructed using a frequency-doubled Gallium Arsenide (GAs) infrared laser or from a diode-pumped Neodymium-doped Yttrium Aluminum Garnet (Nd:YAG) or Neodymium-doped Yttrium Orthovanadate (Nd:YVO4) crystal.

As shown in FIGS. 3A and 3B, a lens 74 is positioned at one end of the housing 68 in the path of the generated laser light for focusing the light onto tissue treatment areas of, for example, 0.5 mm² to 2 mm², and to produce in the treatment areas an energy density in the range of from about 0.01 to about 0.15 joules/mm². The lens 74 may be adjusted to determine depth and area of absorption.

For purposes of appetite suppression key acupuncture/acupressure points are located on the ears, face, lower arm (forearm) and hands. The wand 14 is held against the surface of the tissue with the laser diode 70 contacting the selected acupuncture point to be irradiated with the laser light. Because laser light is coherent, a variable energy density of the light of from about 0.01 to 0.15 joules/mm² is obtained as the light passes through the lens 74 and converges onto each of the small treatment areas.

The present invention has several advantages. For example, by using an In:GaAs or In:GaN diode laser to generate the laser beam energy, the laser source can be made sufficiently small to fit within the hand-held wand 14, thereby obviating the need for a larger, more expensive laser source and the fiber optic cable necessary to carry the laser energy to the treatment tissue. The In:GaAs diode laser can also produce greater laser energy at a higher power dissipation level than lasers of comparable size. Furthermore, construction of the wand 14 including the fins 58 provides for the dissipation from the wand of the heat generated by the laser source. In addition, while the present example illustrated in FIG. 1 only includes one laser wand 14, it should be understood that multiple laser diodes and wands may be used to treat large patients or to treat multiple acupuncture/acupressure points simultaneously.

It is understood that several variations may be made in the foregoing without departing from the scope of the invention. For example, any number of fins 58 may be utilized as long they dissipate sufficient heat from the wand 14 so that the user may manipulate the wand without getting burned. The setting controls 24 may be used individually or in combination and the information displayed on the displays 26 may vary. Other diode laser structures may be utilized to produce the desired effects.

In an alternate embodiment of the present invention, a light emitting diode (LED) is used instead of a laser diode to generate the blue light in order to produce more diffuse light exposure on the acupuncture points.

For purposes of appetite suppression key acupuncture/acupressure points are located on the ears, face, lower arm (forearm) and hands. The surface of the tissue in the region to be treated is irradiated with the laser beam light to produce the desired therapeutic effect.

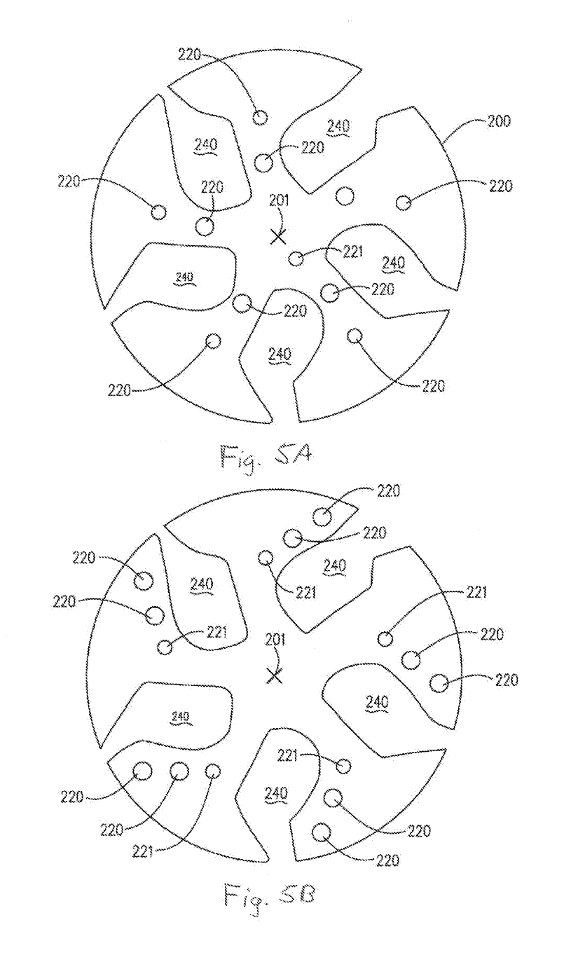

FIG. 4 illustrates an illumination helmet that is used in conjunction with laser stimulation of acupuncture/acupressure points. The helmet 120 contains a plurality of blue light sources that illuminate the head and upper torso of the patient while the acupuncture/acupressure points are concurrently irradiated by a laser source. Blue light therapy has been applied to a variety of maladies including acne, MRSA, depression, sleep disorders, and even liver issues such as neonatal jaundice and Crigler-Najjar Syndrome. Blue light is an important factor for appetite suppression in combination with acupuncture/acupressure points on the face, hands, arms and other places on the body.

The helmet 120 may be supported on a cantilevered support 140 to allow the helmet 120 to be positioned over and about the head of a patient while maintaining a non-contact spacing between the interior of the helmet 120 and the scalp. The helmet 120 is positioned a sufficient distance from the patient's head to allow enough dispersion of the blue light from the light sources to bathe the whole head and parts of the neck and upper torso in blue light. The patient's head may optionally be supported by an external chair having a neck rest (not shown) so that spacing between the scalp and the interior of the helmet 120 may be maintained. The helmet 120 may provide stable support for a cap 200 therein, with the cap 200 being actuated for rotation by a motor 210.

A wiring harness 160 may be provided between the helmet 120 and a controller 180 that provides control and power to components contained within the helmet 120. In the embodiment shown, the wiring harness 160 may be routed through a hollow interior of the cantilevered support 140 for convenience and to protect the wiring harness 160 from snagging or damage. However, the wiring harness 160 may also be attached directly to the helmet 120 by means of a coiled cable, a bundle of bound wires, or other means well known to the art.

The controller 180 may include a power supply 181, a computer 182, an optional magnetic stripe card reader 183, and manual controls (not shown). The power supply 181 may be of standard design having sufficient capacity to power a computer 182, actuate the motor 210 within the helmet 120 and to drive light sources within the cap 200, as will be described presently. The computer 182 may provide control to the motor and light sources and receive direction from manual controls (not shown) associated with the controller 180. The magnetic stripe card reader 183 may be representative of various input devices well known to the art, which allow data to be provided to and received by the computer 182.

It should be understood that the configuration described above is representative of the inventive device and modifications providing the same functionality may be used within the scope of the invention. For example, in some embodiments, the wiring harness 160 may be replaced by a wireless protocol in which the controller 180 may broadcast control information to a receiving unit located in the helmet 120, with the controller 180 and the helmet 120 having their own independent power supplies 181. The magnetic stripe card reader 183 may be substituted with a flash memory card, USB drive, or wireless control to a smart phone or tablet computer or similar device used to input data. Other modifications may be contemplated as being within the scope of the invention.

Controller 180 may be laser control system 10 depicted in FIG. 1, allowing the same underlying system to control both the wand 14 and helmet 120. Alternatively, the helmet 120 and wand 14 may have independent power sources and controllers co-located within the same system.

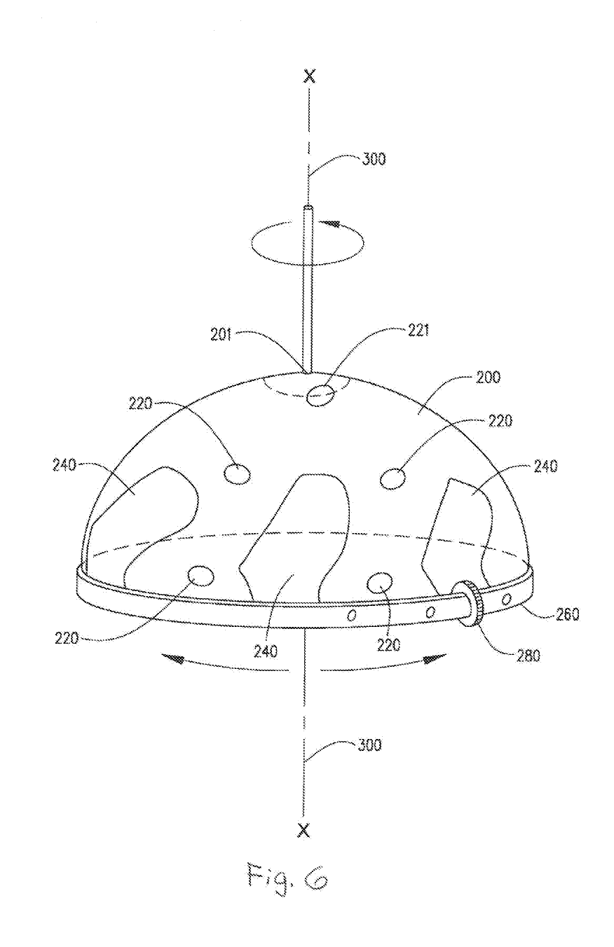

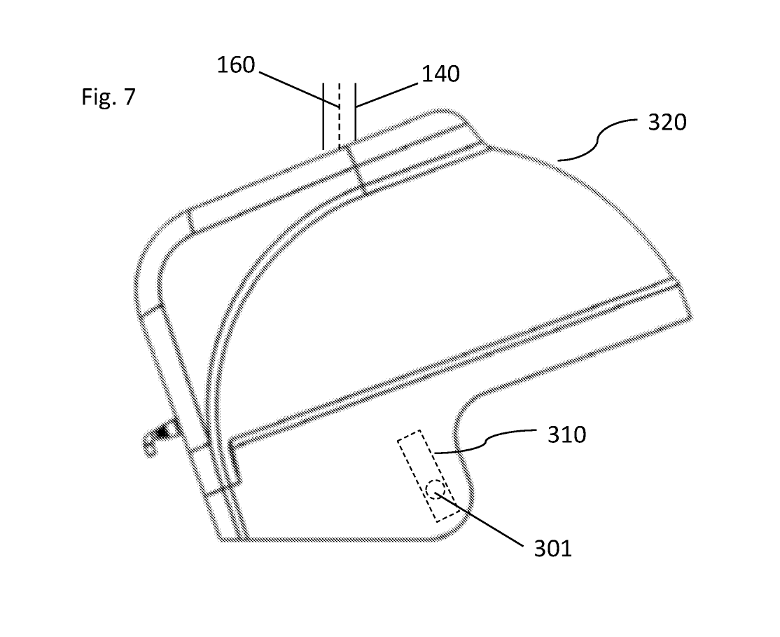

The cap 200 contained within the helmet 120 may be of a generally circular aspect. A flattened pattern for the cap 200 is shown in FIGS. 5A and 5B, which has a center of rotation 201. Cutouts 240 may be removed from the flattened pattern to allow the resulting shape to assume a three-dimensional form as by bending or folding the portions remaining between the cutouts 240. The cap 200 may be formed by folding each portion inwardly in the same direction to form what geometrically is known as a spherical cap (FIG. 6), which is defined as the shape resulting from a plane passing through a sphere. The diodes 220 in the cap 200 may be inwardly directed towards the interior of the cap 200. The cap 200 thus formed may be sized to allow its shape to be position over the patient's head for rotational movement without making contact with the patient's head. The spherical cap may extend so far as to form a geometric hemisphere, but preferably the spherical cap forming cap 200 may typically comprise from one-half to one-third of a hemisphere. Cap 200 may be fabricated of a thin, durable flexible material, which can be formed into the spherical cap shape as shown in FIG. 6.

The diodes 220 in the cap 200 may be Gallium Nitride (GaN) or Indium-doped Gallium Nitride (In:GaN) semiconductor laser diodes producing a low level reactive laser light having a power output level of 100-1000 mw, typically less than 500 mw, and a wavelength ranging from approximately 400-495 nm in the blue region of the electromagnetic spectrum. Alternatively, the blue-light laser diodes 220 can be constructed using a frequency-doubled Gallium Arsenide (GAs) infrared laser or from a diode-pumped Neodymium-doped Yttrium Aluminum Garnet (Nd:YAG) or Neodymium-doped Yttrium Orthovanadate (Nd:YVO4) crystal. Alternatively, the diodes 220 in the cap 200 may be blue light LEDs instead of laser diodes.

Referring now to FIG. 6, an adjustment strap 260 may be provided about the bottom of cap 200, with a knurled adjustment knob 280 to adjust the shape of cap 200 to accommodate various head sizes, in a well-known manner. In another embodiment, the adjustment strap 260 may be overlapped and secured by using a standard hook-and-loop device that is well known to the industry and sometimes marketed under the trademark Velcro®. Other devices for adjusting and securing the strap to accommodate differing head sizes may be used without departing from the scope of the invention.

Cap 200 may be designed for rotation about an axis 300 that passes through the center of rotation 201. Such rotation may be accomplished through any conventional motor means known to the art. The number of diodes 220, the placement of the diodes 220 about the cap 200, the cyclical sequence of rotational movement, and the actuation of the diodes 220 may be design choices that depend upon the areas of the desired light dispersion patter over the head and upper torso.

In the embodiment shown in FIGS. 5A, 5B, and 6, five pairs of circumferentially-spaced diodes 220 may be placed so that they flank cutouts 240 in cap 200. An eleventh diode 221 may be located near center of rotation 201. Although only 11 diodes 220, 221 are shown for illustrative purposes, as many as 20 to 30 single diodes 220 may be placed in cap 200. Additionally and without departing from the scope of the invention, the site for each diode 220 may comprise a cluster of diodes 220, so that the area traversed by the cluster is broader than the area traversed by a single diode 220. It should also be noted that the spacing of diodes 220, 221, as shown in FIGS. 5A, 5B, and 6, is not to scale and is understood to be for illustration purposes only.

FIG. 7 shows an alternate embodiment of the illumination helmet. In this embodiment, in addition to the blue light diodes 220, helmet also includes laser diodes 301 are placed on the side of the inside of the helmet 320 proximal to target acupuncture points on the ears of the patient. Similar to helmet 120, helmet 320 is fed by wiring harness 160 routed through cantilevered support 140.

Similar to diode 70 described above, diodes 301 may be Gallium Arsenide (GaAs) or Indium-doped Gallium Arsenide (In:GaAs) laser diodes having a power output level of 50-500 mw and a wavelength of approximately 1000-2500 nm in the near-infrared region of the electromagnetic spectrum. Alternatively, diodes 301 can be a Gallium Nitride (GaN) or Indium-doped Gallium Nitride (In:GaN) semiconductor diode having a power output level of 100-1000 mw and a wavelength ranging from approximately 400-495 nm in the blue region of the electromagnetic spectrum. The blue-light laser diodes can also be constructed using a frequency-doubled Gallium Arsenide (GAs) infrared laser or from a diode-pumped Neodymium-doped Yttrium Aluminum Garnet (Nd:YAG) or Neodymium-doped Yttrium Orthovanadate (Nd:YVO4) crystal.

The diodes 301 are mounted on tracks 310 inside the helmet 320. In one embodiment, the position of the diodes 301 can be adjusted along the tracks 310 to customize the position of the diodes over the ears of different patients to more accurately irradiate the intended acupuncture/acupressure points on the ears. This embodiment of allows simultaneous laser irradiation of the earlobe acupuncture points and diffuse blue light illumination of the whole head by the helmet 320 without the need for using the wand 14 on the earlobes.

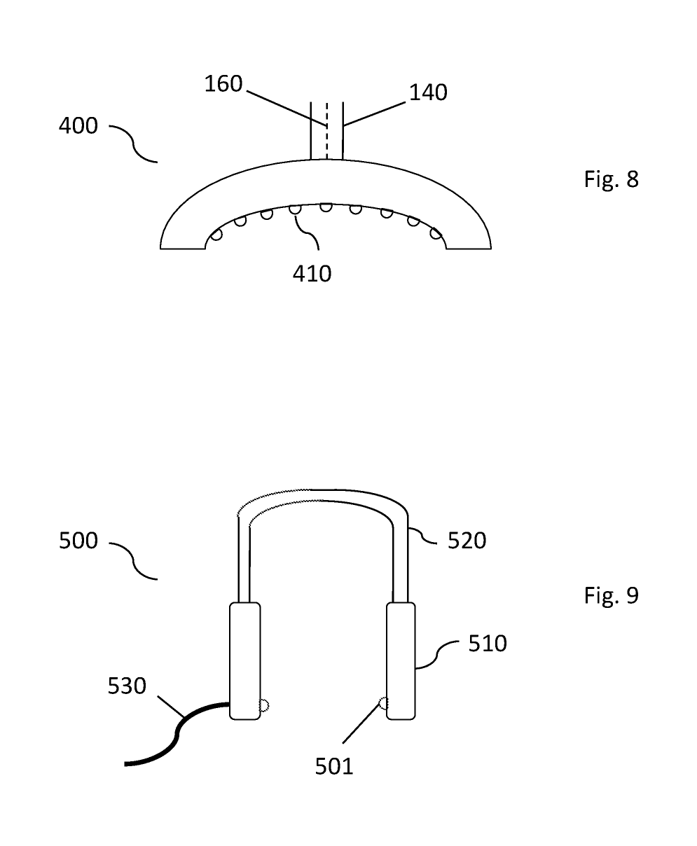

FIG. 8 shows a blue light lamp in accordance with an embodiment of the invention. Similar to helmets 120, 320, lamp 400 is positioned over the head of the patient and is fed by wiring harness 160 routed through cantilevered support 140. Lamp 400 includes a plurality of blue light diodes 410 similar to diodes 220 described above. The lamp 400 is designed to be positioned a distance above the patient's head rather than fit around it like a helmet. This design allows for a more diffuse illumination of the patient's head and torso than the helmets 120 and 320, providing less concentrate illumination of the head but greater exposure to the neck and upper torso.

FIG. 9 shows ear covers containing laser diodes for stimulating acupuncture points on the ears in accordance with an embodiment of the invention. The ear cover laser device 500 includes a separate ear cover 510 for each ear that a patient would wear similar to audio headphones. The inside contact surface of each ear cover 510 includes at least one laser diode 501 that contacts and irradiated acupuncture points on the ear. More specifically, acupuncture points on the earlobes are of particular importance. Similar to audio headphones, the ear covers 510 are held in place on the patient's head by a headband 520, which provides the necessary retaining force to keep the laser diodes 501 in contact with the acupuncture points on the ears.

As with the diode 70 in the wand 14, the diodes 501 in the ear covers 510 may be Gallium Arsenide (GaAs) or Indium-doped Gallium Arsenide (In:GaAs) laser diodes having a power output level of 50-500 mw and a wavelength of approximately 1000-2500 nm in the near-infrared region of the electromagnetic spectrum. Alternatively, diodes 501 can be a Gallium Nitride (GaN) or Indium-doped Gallium Nitride (In:GaN) semiconductor diode having a power output level of 100-1000 mw and a wavelength ranging from approximately 400-495 nm in the blue region of the electromagnetic spectrum. The blue-light laser diodes can also be constructed using a frequency-doubled Gallium Arsenide (GAs) infrared laser or from a diode-pumped Neodymium-doped Yttrium Aluminum Garnet (Nd:YAG) or Neodymium-doped Yttrium Orthovanadate (Nd:YVO4) crystal.

Power and control of the diodes 501 is provided through lead 530, which is coupled to the laser control unit 10 described above in place of or in addition to the wand 14.

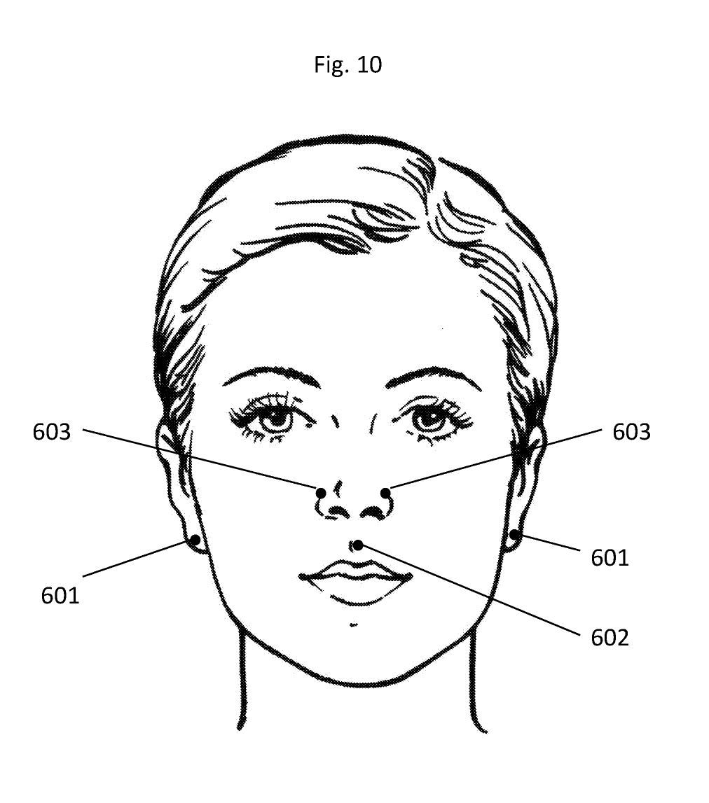

FIG. 10 illustrates the acupuncture/acupressure points on the face and head that are stimulated to suppress appetite in accordance with the invention. These acupuncture points includes the earlobes 601, the upper lip 602 just below the nose, and the sides of the nose 603 at the alar nasal sulcus. In some systems of acupuncture, the earlobe points 601 are known as the TW 17 acupuncture points, the upper lip 602 is known as the GV 26 point, and sides of the nose 603 are known as LI 20.

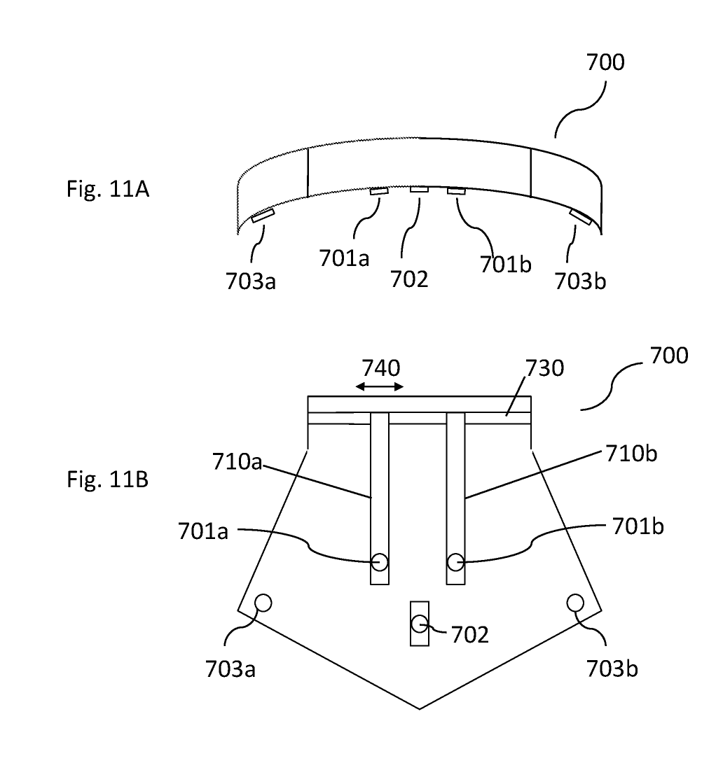

FIG. 11A is a top view of a face cover 700 that stimulates acupuncture points on the face in accordance with the invention. FIG. 11B shows in the inside facing surface of the face cover. The face cover 700 includes a plurality of laser diodes or LEDs 701a, 701b, 702, 703a, and 703b.

When used in conjunction with helmet 120 or helmet 320, the face cover 700 can be detachably mounted to the helmet and receive power and control through wiring harness 160. Alternatively, the face cover 700 may be held in place by an adjustable strap or harness (not shown) and receive power and control through an independent lead from laser controller 10 in place of or in addition to the wand 14.

Similar to diode 70 described above, diodes 701a, 701b, 702, 703a, and 703b may be Gallium Arsenide (GaAs) or Indium-doped Gallium Arsenide (In:GaAs) laser diodes having a power output level of 50-500 mw and a wavelength of approximately 1000-2500 nm in the near-infrared region of the electromagnetic spectrum. Alternatively, diodes can be a Gallium Nitride (GaN) or Indium-doped Gallium Nitride (In:GaN) semiconductor diode having a power output level of 100-1000 mw and a wavelength ranging from approximately 400-495 nm in the blue region of the electromagnetic spectrum. The blue-light laser diodes can also be constructed using a frequency-doubled Gallium Arsenide (GAs) infrared laser or from a diode-pumped Neodymium-doped Yttrium Aluminum Garnet (Nd:YAG) or Neodymium-doped Yttrium Orthovanadate (Nd:YVO4) crystal.

The curvilinear shape of the face cover 700 allows it to hold the laser diodes 701a, 701b, 702, 703a, and 703b in contact with the respective acupuncture points on the face. For example, diodes 701a and 701b are held in contact with acupuncture points on the sides of the nodes and diode 502 is held in contact with the acupuncture point on the upper lip. In one embodiment, the face cover 700 also includes diodes 703a and 703b to stimulate the acupuncture/acupressure points on the ears, depending on which version of the illumination helmet 120, 320 or lamp 400 is used or whether ear cover unit 500 is used. It should be understood that the depiction of face cover 700 in FIGS. 11A and 11B is not to scale.

In one embodiment of the invention, diodes 701a and 701b are mounted on tracks 710a and 710b, respectively and can be repositioned vertically along those tracks for customized positioning for different patients. Tracks 710a, 710b can also be translated horizontally along track 730, as indicated by arrow 740. This allows the nose diodes 701a, 701b to be positioned closer or farther apart from each other for different sized faces and noses.

The face cover 700 offers the advantage of being able to simultaneously stimulate all of the key appetite suppression acupuncture/acupressure points of the face concurrently with the blue light illumination above the head from the helmet without having to use the wand 14 on each individual point. In addition, by prepositioning the laser diodes on the acupuncture/acupressure points, the face cover 700 allows the patient to be treated unattended, taking workload off the care provider. Similarly, ear cover unit 500 also allows stimulation of the ear acupuncture points unattended.

It should be understood that laser glasses or similar protective eyewear should be worn by the patient during treatment with the face cover 700 or any time lasers are used near the face and to protect the eyes from blue light exposure.

The description of the present invention has been presented for purposes of illustration and description, and is not intended to be exhaustive or limited to the invention in the form disclosed. Many modifications and variations will be apparent to those of ordinary skill in the art. The embodiment was chosen and described in order to best explain the principles of the invention, the practical application, and to enable others of ordinary skill in the art to understand the invention for various embodiments with various modifications as are suited to the particular use contemplated. It will be understood by one of ordinary skill in the art that numerous variations will be possible to the disclosed embodiments without going outside the scope of the invention as disclosed in the claims.

I claim:

1. A method for suppressing appetite, comprising:
placing a first illumination device over a patient's head, wherein the first illumination device has a plurality of light sources in a fixed configuration thereon, the configuration selected to illuminate the patient's head, each light source having a dispersion pattern and emitting blue light with a wavelength in the range of 400 nm-495 nm at a power level of 1000 mw or less;
actuating the light sources in the first illumination device to illuminate the patient's head;
selecting at least one acupuncture point on the patient; and
concurrently with illuminating the patient's head with the light sources in the first illumination device, irradiating the selected acupuncture point with coherent laser light from a second illumination device, the laser light having a wavelength in the range of 400 nm-2500 nm at a power level of 500 mw or less.

2. The method according to claim 1, wherein the coherent laser light from the second illumination device has a wavelength of 400 nm-495 nm.

3. The method according to claim 1, wherein the coherent laser light from the second illumination device has a wavelength of 1000 nm-2500 nm.

4. The method according to claim 1, wherein the second illumination device is face cover having a plurality of laser diodes fixed in a configuration for contacting a plurality of respective acupuncture points on the patient's face.

5. The method according to claim 1, wherein the second illumination device is a manipulable wand for contact with the acupuncture point.

6. The method according to claim 1, wherein the second illumination device comprises a pair of ear covers having laser diodes held in contact with respective acupuncture points on the patient's ears.

7. The method according to claim 1, wherein the light sources in the first illumination device are laser diodes.

8. The method according to claim 1, wherein the light sources in the first illumination device are LEDs.

9. The method according to claim 1, wherein the first illumination device is a helmet.

10. The method according to claim 1, wherein the first illumination device is a lamp.

11. A light therapy system for appetite suppression, comprising:
a first illumination device configured to be placed over a patient's head, wherein the first illumination device has a plurality of light sources in a fixed configuration thereon, the configuration selected to illuminate the patient's head, each light source having a dispersion pattern and emitting blue light with a wavelength in the range of 400 nm-495 nm at a power level of 1000 mw or less;
a second illumination device having at least one laser diode for contact with an acupuncture point on the patient, wherein the laser diode is configured to produce coherent laser light having a wavelength in the range of 400 nm-2500 nm at a power level of 500 mw or less; and
at least one controller configured to actuate the second illumination device concurrently with the first illumination device.

12. The system according to claim 11, wherein the coherent laser light from laser diode of the second illumination device has a wavelength of 400 nm-495 nm.

13. The system according to claim 11, wherein the coherent laser light from the laser diode of the second illumination device has a wavelength of 1000 nm-2500 nm.

14. The system according to claim 11, wherein the second illumination device is face cover having a plurality of laser diodes fixed in a configuration for contacting a plurality of respective acupuncture points on the patient's face.

15. The method according to claim 11, wherein the second illumination device is a manipulable wand for contact with the acupuncture point.

16. The method according to claim 11, wherein the second illumination device comprises a pair of ear covers having laser diodes held in contact with respective acupuncture points on the patient's ears.

17. The method according to claim 11, wherein the light sources in the first illumination device are laser diodes.

18. The method according to claim 11, wherein the light sources in the first illumination device are LEDs.

19. The method according to claim 11, wherein the first illumination device is a helmet.

20. The method according to claim 11, wherein the first illumination device is a lamp.
